(19) United States
(12) Patent Application Publication
(10) Pub. No.: US 2015/0329857 A1
(43) Pub. Date: Nov. 19, 2015

(54) RNA INTERFERENCE TO ACTIVATE STEM CELLS

(71) Applicant: Universite Pierre et Marie Curie (PARIS 6), Paris (FR)

(72) Inventors: David Sassoon, Paris (FR); Giovanna Marazzi, Paris (FR); Vanessa Besson, Marly le Roi (FR)

(73) Assignee: Universite Pierre et Marie Curie (PARIS 6), Paris (FR)

(21) Appl. No.: 14/367,328

(22) PCT Filed: Dec. 28, 2012
(86) PCT No.: PCT/EP2012/077034

§ 371 Date: Jun. 20, 2014

(30) Foreign Application Priority Data
Dec. 28, 2011 (EP) 11306796.1

(51) Int. Cl. C12N 15/113

(52) U.S. Cl. CPC C12N 15/113; C12N 2310/14; C12N 2310/531

(57) ABSTRACT

The present invention relates to the use of interferent RNAs that silence PW1 expression in order to activate adult stem cells in vitro or in vivo, in particular in the context of regenerative medicine.

The present invention relates to selective knockdown of PW1 expression by RNA interference to modulate mammalian stem cells in vivo and in vitro, which modulation is particularly useful in the context of tissue repair and regenerative medicine.

### BACKGROUND OF THE INVENTION

Stem cells are defined by the ability to continuously self-renew and produce the differentiated progeny of the tissue of their location. Stem cells are a small percentage of the total cellularity. For instance, in the small intestine there are perhaps up to 10 stem cells near the bottom of the crypt out of a total crypt population of less than 300 cells. In skeletal muscle, satellite (stem) cells comprise about 5% of all nuclei, but in the bone marrow the multi-potential hematopoietic stem cell is much rarer, with a frequency of perhaps 1 in 10,000 amongst all bone marrow cells. Considerable overlap exists between different putative organ specific stem cells in their repertoire of gene expression, often related to self-renewal, cell survival and cell adhesion. Stem cells have been used routinely for over three decades to repair several tissues and organs damaged by injury or disease that are amenable to such approaches including most notably bone marrow (Fernand et al, 1989) and skin (Green et al, 1989). Adult stem cells are critical for tissue homeostasis and wound repair and reside within specific niches that preserve proliferative and regenerative potential and as such, serve as promising targets for small-molecule or biological therapeutics aimed at increasing regenerative potential in vivo.

The inventors had previously isolated PW1 in a screen for putative regulators of early muscle stem cells. They found that PW1 was expressed during early embryogenesis and strongly expressed in all early lineages declining during late fetal development and was confined to muscle stem cells in the adult. Moreover, studies with PW1 led to the elucidation of a novel population of resident stem cells in adult skeletal muscle. The generation of a PW1 reporter mouse has allowed to easily track endogenous PW1 expression leading to the observation that PW1 is expressed in all adult stem cells.

### SUMMARY OF THE INVENTION

The present invention provides an interferent RNA that silences PW1/PEG3 for use in activating adult stem cells in vivo or in vitro.

In particular, the interferent RNA may be selected from the group consisting of a siRNA, a shRNA, an antisense RNA, and a miRNA.

In a particular embodiment, the interferent RNA is a siRNA that comprises a sense RNA strand and a complementary antisense RNA strand which together form an RNA duplex, wherein the sense RNA strand comprises or consists of sequence SEQ ID NO:3.

In another particular embodiment, the interferent RNA is a shRNA, preferably carried by a lentivirus vector, which shRNA comprises or consists of SEQ ID NO:14.

The invention provides an interferent RNA that silences PW1/PEG3 for use in treating a patient afflicted with a disease treatable by regenerative medicine, including cardiac failure, bone marrow disease, skin disease, burns, degenerative disease such as diabetes, Alzheimer's disease, Parkinson's disease, and cancer.

The invention further provides a method for fighting against skin aging in a subject, which method comprises administering the subject with an interferent RNA that silences PW1/PEG3, as defined herein.

A further subject of the invention is a method for preventing hair loss or promoting hair growth in a subject, which method comprises administering the subject with an interferent RNA that silences PW1/PEG3, as defined in herein.

The invention further provides an in vitro method for activating adult stem cells, which method comprises contacting adult stem cells or a tissue comprising adult stem cells with an interferent RNA that silences PW1/PEG3, as defined herein.

### LEGENDS TO THE FIGURES

FIG. 1 shows a map of a lentiviral vector (named Pw1shRNA) that expresses a shRNA under the control of the human promoter for H1 that silences PW1 (SEQ ID NO: 12 corresponding to the following reference from Sigma: TRCN0000075397NM_{—}008817.2-4305s1c1, named shPW1 in the map).

FIG. 2 shows immunofluorescence of epidermal stem cells infected with PW1shRNA (down panel) or with a GFP-control lentivirus (upper panel) BF: Bright field, Nuclei were counterstained with DAPI (blue), anti-PW1 antibodies (Relaix et al, 1998) immunostained cells in red, GFP emits in green.

FIG. 3 is a graph showing results of immunofluorescence on neural stem cells, that are transfected, with 20 nM or 50 nM scrambled siRNA (Ambion) or with a PW1 siRNA (Ambion, siRNA ID #s71469). Anti-PW1 antibody described in Relaix et al, 1998 was used and nuclei were counterstained with DAPI.

FIG. 4 shows the activation of hair growth cycle by targeting specifically stem cells. Histological sections of skin were obtained 4 weeks after injection of control shRNA or Pw1 shRNA.

FIG. 5 shows the activation of cell cycle using Pw1shRNA, by immunofluorescence staining of skin sections using control lentivirus or Pw1shRNA. Stem cells were immunostained using K15 marker (red), proliferation was revealed using Ki67 (green). Nuclei were counterstained with DAPI (blue).

FIG. 6 shows that a PW1 siRNA (Ambion, siRNA ID #s71469) induces increase in muscle stem cells proliferation. Cells were plated at the same original density and as shown by staining with DAPI (nuclei).

### DETAILED DESCRIPTION OF THE INVENTION

The inventors have now shown that interference with PW1 function leads to stem cell proliferation.

The invention thus provides interferent RNA that silences PW1, for use in activating stem cells.

Pw1/Peg3

Pw1/Peg3 (“paternally expressed gene 3”), herein designated as “Pw1”, is a maternally imprinted gene that is expressed primarily during embryogenesis and in adult ovary, testis, muscle, and brain in mouse. In the present invention, the term “Pw1” or “Peg3” means the mouse Pw1 gene or the orthologous gene in any other animal species, in particular in humans.

Mammalian imprinting regulates growth and the establishment of parental nurturing behaviors, but the detailed molecular mechanisms by which this occurs are incompletely known. Pw1 mediates cell stress and pro-survival pathways in vitro, as well as muscle atrophy and stem cell number in vivo. Kim et al. (2000) mapped the mouse Pw1/Peg3 gene to proximal chromosome 7 and determined that the gene contains 13 exons, the last 4 of which originated from the ancestral ZIM2 gene (Kim et al., 2000). The initiation codon is located in exon 3. Because imprinting is generally conserved among mammals, and imprinted domains generally encompass several adjacent genes, expression patterns and chromosomal environment of the human counterpart of Peg3 was of interest. Kim et al. (1997) localized the human PW1/PEG3 gene approximately 2 Mb proximal to the telomere of 19q, within a region known to carry large numbers of tandemly clustered Kruppel-type zinc finger-containing (ZNF) genes (Kim et al., 1997).

The protein encoded by the Pw1 gene is named the “PW1 protein”. This protein has been conserved during evolution. PW1 protein sequence identity is estimated at 63.9% between human and mouse. In the present invention, the term “PW1” means the mouse Pw1 protein or the orthologous gene in any other animal species, in particular in humans.

Mouse PW1 protein sequence is shown as SEQ ID NO:1 (also available as NP_{—}032843.2), and human PW1 protein sequence is shown as SEQ ID NO:2 (also available as NP_{—}001139656.1, NP_{—}001139658.1, NP_{—}001139659.1, NP_{—}006201.1).

RNA Interference:

Various means for RNA interference may be used. In the context of the present invention, RNA interference (RNAi) includes small nucleic acid molecules, such as micro RNA (miRNA), short-hairpin RNA (shRNA) and/or short or small interfering RNA (siRNA). Antisense RNAs are further encompassed.

Preferred molecules capable of mediating RNA interference advantageously down regulate at least 60%, preferably at least 70%, preferably at least 80%, even more preferably at least 90%, of the target protein expression.

Preferably the siRNA is used in form of synthetic RNA duplexes (ds-siRNAs), i.e, the siRNA is a siRNA duplex comprised of a sense strand homologue to the target and an antisense strand that binds to the target mRN). However single stranded siRNAs (ss-siRNA) was be of use also.

A siRNA according to the invention is a small double stranded RNA with sense and antisense strands paired by Watson-Crick bonds, and in which the sequence of the sense strand consists of or comprises a fragment of 14 to 30, advantageously 15 to 29, 16 to 28, 17 to 27, 18 to 25, 18 to 23, or 18 to 21 contiguous nucleotides of the nucleotide sequence of PW1/PEG3.

It is known that siRNAs with a sequence composed of 30 to 50% of guanines and cytosines are more effective than sequences with a higher proportion of guanines and cytosines. Therefore the siRNAs according to the invention advantageously have a sequence composed of 30 to 50% of guanines and cytosines.

Is should be understood that a siRNA according to the invention can equally comprise two complementary single stranded RNA molecules, or a single single stranded RNA molecule in which two complementary portions are paired by Watson-Crick bonds and are linked covalently on one side by a hairpin type structure (this is more specifically known as shRNA for “short hairpin RNA”), which can be considered as a subclass of siRNA. Throughout the description, the term siRNA should be understood in its broad sense including shRNAs, unless otherwise indicated. In an advantageous embodiment, a siRNA according to the invention comprises two complementary single stranded RNA molecules. In another advantageous embodiment, a siRNA according to the invention comprises or consists of a single molecule of single stranded RNA in which two complementary portions are paired by Watson-Crick bonds and are linked covalently on one side by a hairpin type structure, that is to say it is a shRNA.

It is preferable that the target nucleotide sequence and the sequence complementary thereto contained in the siRNA be completely complementary to each other. However, in the presence of a base mutation at a position apart from the center of the siRNA, the cleavage activity by RNA interference is not completely lost, but a partial activity can remain. On the other hand, a base mutation in the center of the siRNA has a major influence to the extent that it can extremely reduce the mRNA cleavage activity by RNA interference.

Moreover, the sense and/or antisense RNA strands can further comprise a 3′ overhang fragment of 2 to 4 nucleotides, in particular when a siRNA according to the invention comprises two complementary single stranded RNA molecules. The expression “3′ overhang fragment of 2 to 4 nucleotides” as used herein is understood to mean the presence in at least one strand of the RNA duplex of 2 to 4 nucleotides not paired with the complementary strand at the 3′ distal end of said strand. The nucleotides used in the 3′ overhang fragment can be natural nucleotides (ribonucleotides or deoxyribonucleotides), or modified nucleotides such as LNA (Locked Nucleic Acid) which comprises a methylene bridge between the 2′ and 4′ positions of the ribose (Soutschek et al. 2004). The 3′ overhang fragment can also undergo all types of chemical modification described in the following paragraph for the sense RNA strand and/or the antisense RNA strand of a siRNA according to the invention. Advantageously, the 3′ overhang fragment consists of 2 nucleotides. In this case, the preferred sequences for the 3′ overhang fragment are “TT” (where T represents deoxythymidine) or “UU” (where U represents uracil). Equally advantageously, both complementary strands of a siRNA according to the invention comprise a 3′ overhang fragment. In this case, the length and the sequence of the two 3′ overhang fragments can be identical or different. Advantageously, both complementary strands of a siRNA according to the invention each comprise the same 3′ overhang fragment of 2 nucleotides with the sequence “TT”.

Examples of an antisense nucleic acid capable of specifically suppressing the expression of PW1 include: A) a nucleic acid comprising a nucleotide sequence complementary to the nucleotide sequence of an mRNA (mature mRNA or initial transcription product) that encodes PW1 or a partial sequence thereof having 12 bases or more in length, (B) a nucleic acid comprising a nucleotide sequence having 12 bases or more in length that is hybridizable specifically with an mRNA (mature mRNA or initial transcription product) that encodes PW1 in cells of an animal (preferably human) which is a the subject of treatment, and being capable of inhibiting the translation into the PW1 polypeptide in a hybridized state, and the like.

The length of the portion that hybridizes with the target mRNA in the antisense nucleic acid is not particularly limited, as far as the expression of PW1 can specifically be suppressed; the length is generally about 12 bases or more, and up to the same length as the full-length sequence of the mRNA (mature mRNA or initial transcription product). Taking into account hybridization specificity, the length is preferably about 15 bases or more, more preferably 18 bases or more. Taking into account the ease of synthesis, antigenicity issues and the like, the length of the portion that hybridizes with the target mRNA is generally about 200 bases or less, preferably about 50 bases or less, more preferably about 30 bases or less. Hence, the length of the portion that hybridizes with the target mRNA is, for example, about 12 to about 200 bases, preferably about 15 to about 50 bases, more preferably about 18 to about 30 bases.

The target nucleotide sequence for the antisense nucleic acid is not particularly limited, as far as the expression of PW1 can specifically be repressed or suppressed; the sequence may be the full-length sequence of an mRNA (mature mRNA or initial transcription product) of PW1 or a partial sequence thereof (e.g., about 12 bases or more, preferably about 15 bases or more, more preferably about 18 bases or more), or an intron portion of the initial transcription product.

The nucleotide sequence of the portion that hybridizes with the target mRNA in the antisense nucleic acid varies depending on the base composition of the target sequence, and has an identity of generally about 90% or more (preferably 95% or more, most preferably 100%) to the complementary sequence for the target sequence so as to be capable of hybridizing with the mRNA of PW1 under physiological conditions.

Furthermore, the antisense nucleic acid may be one not only capable of hybridizing with the mRNA or initial transcription product of PW1 to inhibit the translation, but also capable of binding to the PW1 gene, which is a double-stranded DNA, to form a triplex and inhibit the transcription into mRNA.

Furthermore, in an interferent RNA according to the invention, such as a siRNA or an antisense, the sense RNA strand and/or the antisense RNA strand can also comprise at least one chemical modification in their sugar portions, their nucleobase portions or their internucleotide backbone. Such modifications can notably make it possible to inhibit the breakdown of siRNAs by nucleases in vivo. All chemical modifications that enable the improvement of the stability and in vivo bioavailability of siRNAs according to the invention are thus included in the scope of the invention. Among the advantageous modifications to the sugar portions, mention can be made notably of modifications taking place in position 2′ of ribose, such as 2′-deoxy, 2′-fluoro, 2′-amino, 2′-thio, or 2′-O-alkyl, and particularly 2′-O-methyl, replacing the normal 2′-OH groups on the ribonucleotides, or the presence of a methylene bridge between positions 2′ and 4′ of ribose (LNA). Concerning nucleobases, it is possible to use modified bases, such as notably 5-bromo-uridine, 5-iodo-uridine, N.sup.3-methyl-uridine, 2,6-diaminopurine (DAP), 5-methyl-2′-deoxyCytidine, 5-(1-propynyl)-2′-deoxy-Uridine (pdU), 5-(1-propynyl)-2′-deoxyCytidine (pdC), or bases conjugated with cholesterol. Lastly, advantageous modifications of the internucleotide backbone include replacing the phosphodiester groups in the backbone by phosphorothioate, methylphosphonate, phosphorodiamidate groups, or using a backbone composed of N-(2-aminoethyl)-glycine units linked by peptide bonds (PNA, Peptide Nucleic Acid). The various modifications (base, sugar, backbone) can obviously be combined to give modified nucleic acids of the morpholino type (bases fixed to a morpholine ring and linked by phosphorodiamidate groups) or PNA (bases fixed to N-(2-aminoethyl)-glycine units linked by peptide bonds).

Interferent RNAs, such as siRNAs, according to the invention are “isolated”, which means that they are not in a natural state but have been obtained by any means involving human intervention. Notably, siRNAs according to the invention can have been obtained by purification of siRNAs that already exist, by chemical synthesis, by amplification of the desired nucleotide sequences by a polymerase chain reaction (PCR), or by recombinant synthesis. Many companies also offer customised siRNA synthesis, notably companies such as Eurogentec, Ambion, Dharmacon, or Qiagen.

An siRNA and antisense nucleic acid capable of specifically suppressing the expression of PW1 can be prepared by determining the target sequence on the basis of an mRNA sequence or chromosomal DNA sequence of PW1, and synthesizing a nucleotide sequence complementary thereto using a commercially available automated DNA/RNA synthesizer (Applied Biosystems, Beckman and the like). The siRNA can be prepared by separately synthesizing a sense strand and an antisense strand using an automated DNA/RNA synthesizer, and denaturing the strands in an appropriate annealing buffer solution at about 90° C. to about 95° C. for about 1 minute, and then performing annealing at about 30° C. to 70° C. for about 1 to about 8 hours. A longer double-stranded polynucleotide can be prepared by synthesizing complementary oligonucleotide strands in a way such that they overlap with each other, annealing the strands, and then performing ligation with a ligase.

siRNA oligonucleotides designed to silence PW1 gene are commercially available, e.g. from Ambion.

In a preferred embodiment, the invention makes use of a siRNA that shows a nucleotide sequence selected from the group consisting of SEQ ID NO: 3 to SEQ ID NO:8, preferably in duplex form.

The siRNA molecules may be either synthesized or produced by cleavage of corresponding shRNAs by DICER. Such shRNAs can be produced from vectors comprising corresponding nucleic acid sequences.

Other siRNA sequences that silence PW1 can be easily designed by any person skilled in the art.

Examples of siRNAs of interest are shown below:

| siRNA | Sense | Antisense |
|---|---|---|
| Ambion, | 5′GAG UCG CAG UCA | 5′AAU CGA UUG ACU |
| siRNA ID# | AUC GAU Utt 3′ | GCG ACU Cag 3′ |
| s71469 | (SEQ ID NO: 3) | (SEQ ID NO: 4) |
| Ambion, | 5′CCA UGG UAG AGG | 5′UGA GUU GCC UCU |
| siRNA ID# | CAA CUC Att 3′ | ACC AUG Gat 3′ |
| s71468 | (SEQ ID NO: 5) | (SEQ ID NO: 6) |
| Ambion, | 5′GAC CAG CUG UAU | 5′UUA CGG AAU ACA |
| siRNA ID# | UCC GUA Att 3′ | GCU GGU Ctt 3′ |
| s71467 | (SEQ ID NO: 7) | (SEQ ID NO: 8) |

Examples of shRNAs of interest are shown below:

SIGMA
shRNA 3′utr

| TRCN000007 | CCGGCCTCTTAGATAGTCCTGTGAACTCGAGTTCACAGG |
|---|---|
| 5393 | ACTATCTAAGAGGTTTTTG (SEQ ID NO: 9) |
| TRCN000007 | CCGGCCCTAATGACAAGCTGAAATTCTCGAGAATTTCAG |
| 5395 | CTTGTCATTAGGGTTTTTG (SEQ ID NO: 10) |
| TRCN000007 | CCGGGCCGAGTCATACCAGAATGTTCTCGAGAACATTCT |
| 5396 | GGTATGACTCGGCTTTTTG (SEQ ID NO: 11) |
| TRCN000007 | CCGGCCACTGTACGAATGCAAAGATCTCGAGATCTTTGC |
| 5397 | ATTCGTACAGTGGTTTTTG (SEQ ID NO: 12) |
| TRCN000007 | CCGGCCTCCATTTATATCCCAGATACTCGAGTATCTGGG |
| 5394 | ATATAAATGGAGGTTTTTG (SEQ ID NO: 13) |

Sequence ACTGTACGAATGCAAAGAT (SEQ ID NO: 14) is the portion specific to PW1 within SEQ ID NO:12.

Vectors:

In a preferred embodiment, the interferent RNA is carried by an expression vector. In the expression vector, the above-described siRNA or antisense nucleic acid or a nucleic acid (preferably DNA) that encodes the same has been operably linked to a promoter capable of exhibiting promoter activity in cells of a mammal (preferably human).

Any promoter capable of functioning in the cells of the mammal which is the subject of administration can be used. Useful promoters include pol I promoters, pol II promoters, pol III promoters and the like. Specifically, viral promoters such as the SV40-derived initial promoter and cytomegalovirus LTR, mammalian constitutive protein gene promoters such as the beta-actin gene promoter, RNA promoters such as the tRNA promoter, and the like are used. More particularly human promoter for H1 may be used, in particular for controlling shRNA expression.

When the expression of an siRNA is intended, it is preferable that a pol III promoter be used as the promoter. Examples of the pol III promoter include the U6 promoter, H1 promoter, tRNA promoter and the like.

At least three methods to generate RNAi-mediated gene silencing in vivo are known and usable in the context of the present invention (Dykxhoorn et al., 2003 for review):

siRNAs with a single sequence specificity can be expressed in vivo from plasmidic or viral vectors using:
Tandem polymerase III promoter that expresses individual sense and antisense strands of the siRNAs that associate in trans;
a single polymerase III promoter that expresses short hairpin RNAs (shRNAs)
a single polymerase II promoter that expresses an imperfect duplex hairpin RNA (pre-miRNA) which is processed by DICER giving rise to a mature miRNA.

The expression vector preferably contains a transcription termination signal, i.e., a terminator region, downstream of the above-described polynucleotide or nucleic acid that encodes the same. Furthermore, a selection marker gene for selection of transformed cells (e.g., genes that confer resistance to drugs such as tetracycline, ampicillin, and kanamycin, genes that compensate for auxotrophic mutations, and the like) can further be included.

Although there is no limitation on the choice of expression vector useful in the present invention, suitable vectors for administration to mammals such as humans include viral vectors such as retrovirus, lentivirus, adenovirus, and adeno-associated virus. Adenovirus, in particular, has advantages such as very high gene transfer efficiency and transferability to non-dividing cells. Because the integration of transgenes into host chromosome is extremely rare, however, the gene expression is transient and generally persists only for about 4 weeks. Considering the persistence of therapeutic effect, it is also preferable to use adeno-associated virus, which offers a relatively high efficiency of gene transfer, which can be transferred to non-dividing cells as well, and which can be integrated into chromosomes via an inverted terminal repeat (ITR).

In a preferred embodiment, the interferent RNA is preferably a shRNA carried by a lentiviral vector that generates lentiviral transduction particles in packaging cell lines.

Activating Stem Cells

The interferent RNA of the invention is useful to activate adult stem cells, i.e. to trigger or enhance their proliferation.

In the context of the present invention, the term “adult stem cells” include induced pluripotent stem cells, which are artificially derived from a non-pluripotent cell, typically an adult somatic cell.

The cells may belong to any tissue, including blood, bone marrow, hematopoietic system, skin, hair follicle, muscle, nervous system, heart, intestine, thymus, pancreas, testis, eye, kidney, liver, lung, spleen, tongue, bones, dental pulp, breast, ovaries, uterus, and placenta.

The interferent RNA of the invention may be used in vitro or in vivo.

In one embodiment, the RNA may be used in vitro to propagate stem cells for regenerative therapy. Stem cells may be expanded in vitro and then directly administered into a patient.

In another embodiment, the interferent RNA may be administered to a patient, to propagate stem cells in vivo.

The interferent RNA of the invention is of interest in tissue repair, which may be useful in treating neurodegenerative diseases, including stroke and Alzheimer's disease, in spinal cord injury, as well as cardiovascular diseases, in particular myocardial infarction. Another field of regenerative medicine is skin repair, in particular for burns or genetic diseases. It may be particularly using in preventing aging, or slowing-down cell aging, in particular skin aging. The interferent RNA of the invention may be also useful in promoting hair growth.

The patient or subject may be any human or non-human animal, preferably a mammal, including rodent, sheep, goats, cattle, horses, dogs, cats, primates.

Pharmaceutical or Cosmetic Compositions:

The invention further relates to interferent RNAs s according to the invention, for use as drugs or cosmetics.

Another object of the invention is a composition comprising at least one interferent RNA according to the invention and an acceptable carrier. The term “acceptable carrier” as used herein is understood to mean any cosmetologically or pharmacologically acceptable carrier known to those skilled in the art.

Any conventional route of administration is encompassed, including oral, pulmonary, intraperitoneal (ip), intravenous (iv), intramuscular (im), subcutaneous (sc), transdermal, buccal, nasal, sublingual, ocular, rectal and vaginal. In addition, administration directly to the tissue where proliferation of adult stem cells is desired may be contemplated. The interferent RNA can be formulated by methods known in the art. Compositions for the oral, rectal, parenteral or local application can be prepared in the form of tablets, capsules, granulates, suppositories, implantages, sterile injectable aqueous or oily solutions, suspensions or emulsions, aerosols, salves, creams, or gels, retard preparations or retard implantates. The interferent RNA may also be administered by implantable dosing systems or by infusion.

In a particular embodiment, the composition according to the invention is intended for cosmetic and/or therapeutic treatment of the skin or the hair, and is thus advantageously administered topically.

A composition for topical application according to the invention can be formulated in any galenic form habitually used for topical application, such as for example in the form of an aqueous solution, a white or colored cream, an ointment, a milk, a lotion, a gel, a salve, a serum, a paste, an oil in water or water in oil emulsion, or a foam. It is possible to apply it to the skin in an aerosol form. It can also be presented in the form of a solid, either powdery or not, for example in the form of a stick. When intended for administration to the hair or hair scalp, the composition may be in form of a shampoo, a lotion, a gel, or a foam, for instance.

It will be readily apparent to those skilled in the art that any dose or frequency of administration that provides the therapeutic effect is suitable for use in the present invention. For example, the therapeutically effective amount may be from about 10 nM to 100 nM in vitro and from about 0.01 μg/g of tissue to about 25 μg/g of tissue in vivo.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate.

The pharmaceutical composition may further contain a reagent for nucleic acid transfer in order to promote the transfer of the nucleic acid into a cell.

Useful nucleic acid transfer reagents include cationic lipids such as lipofectin, lipofectamine, lipofectamine RNAiMAX, invivofectamine, DOGS (transfectam), DOPE, DOTAP, DDAB, DHDEAB, HDEAB, polybrene, and poly(ethylenimine) (PEI). When a retrovirus is used as the expression vector, retronectin, fibronectin, polybrene and the like can be used as transfer reagents.

Physical techniques can also enhance RNA uptake at a specific tissue site using electroporation, pressure, mechanical massage, etc. Terminal modification of RNAs can enhance their resistance to degradation by exonucleases in serum and tissue. Moreover, modification with a suitable ligand can achieve targeted delivery. Several types of carrier for drug delivery have been developed for RNA in addition to traditional cationic liposome and cationic polymer systems. Ultrasound and microbubbles or liposomal bubbles have also been used in combination with a carrier for RNA delivery. New materials with unique characteristics such as carbon nanotubes, gold nanoparticles, and gold nanorods have attracted attention as innovative carriers for RNA. For a recent review, see Higuchi et al, 2010.

A composition according to the invention can further comprise any kind of vehicle known to those skilled in the art making it possible to improve the delivery and the bioavailability of an interferent RNA according to the invention.

Particular vehicles that can be used with interferent RNA comprise notably liposomes and peptides able to cross the cell membrane (known as CPP for “Cell-Permeable Peptides”). The expression CPP as used herein is understood to mean peptides able to be internalised and then reach the cytoplasmic and/or nuclear compartments of living cells. Examples of such CPPs include the peptides Penetratine, Transportan, Tat, MAP and SynB1. Other carriers may be used in the form of polymeric nanoparticles or microparticles, liposomes and micelles (Allen, et al 2004; Farokhzad, et al 2009).

In a particular embodiment, the RNA is formulated in a nanoparticle. Generally speaking, nanoparticle-based delivery systems are delivery reagents that compact siRNA into particles in the optimal size range of hundreds of nanometers that are on the order of 100,000,000 Daltons in mass. The predominant packaging strategy is to utilize the anionic charge of the siRNA backbone as a scaffold for electrostatic interaction with the delivery reagent. Cationic lipids, cationic polymers, and cationic peptides, which can advantageously be combined with cholesterol, are used to engage the negatively charged phosphodiester backbone and organize large numbers of RNA molecules into nanoparticle structures prior to cellular treatment in vitro or systemic administration in vivo (Whitehead et al., 2009. See also e.g. WO 2010/080724; US 2006/0240554 and US 2008/0020058).

Beyond cationic motifs required for interferent RNA nanoparticle formation, additional motifs are applied to the delivery reagent. A large variety of lipids, cell targeting ligands, antibodies, and cell penetrating peptides, to list a few, can be covalently tethered to the cationic packaging motifs so that the resulting nanoparticles that are formed will have cellular delivery properties (Whitehead et al., 2009).

Natural particulates such as pathogens (bacteria, viral vector systems) and human cells can also be used to deliver nucleic acid fragments (for review see Yoo et al, 2011).

Other teams have developed useful polymer drug conjugates that allow specific cell targeting and provide dose control, high release efficiency and low toxicity such as hyaluronan (HA) based injectable hydrogel (Oommen et al, 2009).

The invention also relates to the use of at least one interferent RNA or of a composition according to the invention as a cosmetic or pharmaceutical agent to activate adult stem cells.

The interferent RNA may be used to activate adult stem cells in vivo or in vitro.

The interferent RNA may be useful when activation or expansion of stem cells is desired, when treating a particular disease. Such disease may comprise a disease treatable by regenerative therapy, including cardiac failure, bone marrow disease, skin disease, burns, degenerative disease such as diabetes, Alzheimer's disease, Parkinson's disease, etc and cancer.

It is thus described a method for treating such disease by activating adult stem cells through administration of at least an interferent RNA described herein.

The invention also relates to a method of cosmetic treatment for fighting against skin aging or hair loss, comprising administering a composition according to the invention, preferably topically.

The Examples and Figures illustrate the invention without limiting its scope.

### EXAMPLES

### Example 1

### Decrease of PW1 Protein Using Interferent RNA

1.1. Decrease of PW1 Protein Using a shRNA Expressing Lentivirus

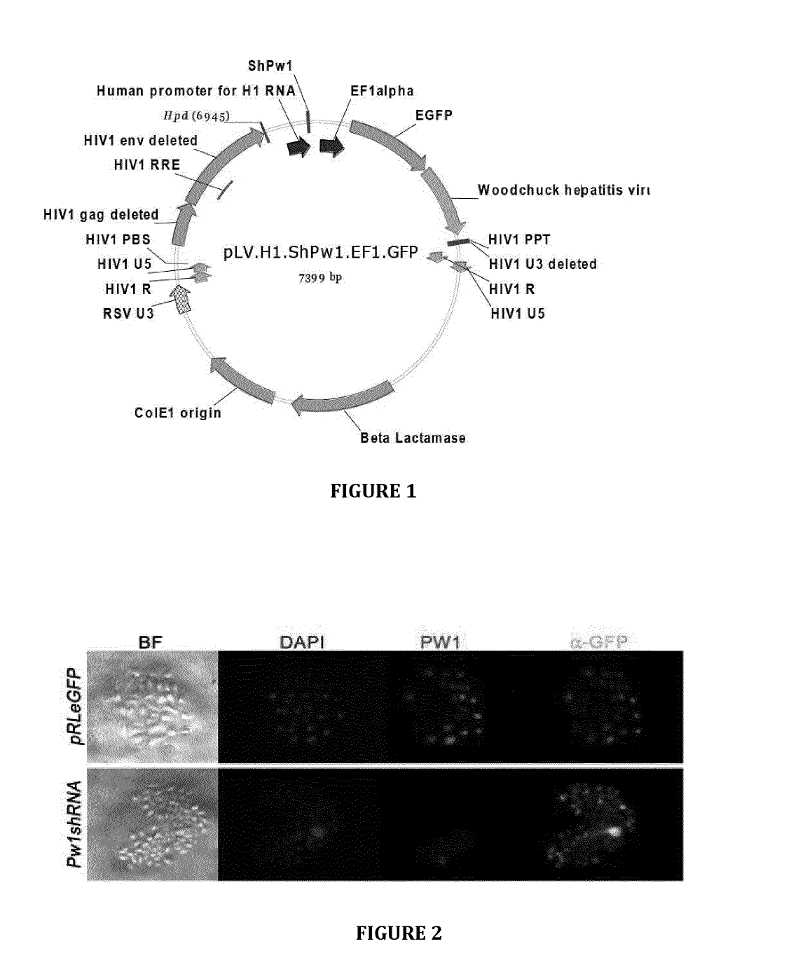

The inventors have used a lentiviral particle provided by Vectalys, and expressing a shRNA (SEQ ID NO: 12 corresponding to the following reference from Sigma: TRCN0000075397NM_{—}008817.2-4305s1c1) that effectively silences the expression of PW1 protein. The map of the lentiviral particle is shown in FIG. 1.

Results have been obtained with adult bulge-derived skin stem cells. These epidermal stem cells, infected with this Pw1 shRNA lentivirus or a GFP-control, lentivirus were subjected to immunofluorescence experiments (FIG. 2). 20,000 freshly isolated skin stem cells derived from adult bulge were infected with the Pw1shRNA (MOI 200) in 50 μl growing medium deprived of serum for 30 min at 37° C. Cells were plated in growing medium containing serum and were subjected to immunofluorescence experiments.

Pw1shRNA lentivirus infection of freshly harvested skin stem cells resulted in a higher frequency of clonal colony formation and that the resultant colonies are larger. When the colonies were assayed directly, a decreased level of PW1 expression was observed.

These results demonstrate that a reduction of PW1 expression by interferent RNA ‘mobilizes’ the stem cells through a stimulation of cell proliferation.

1.2. Decrease of PW1 Protein Using siRNA

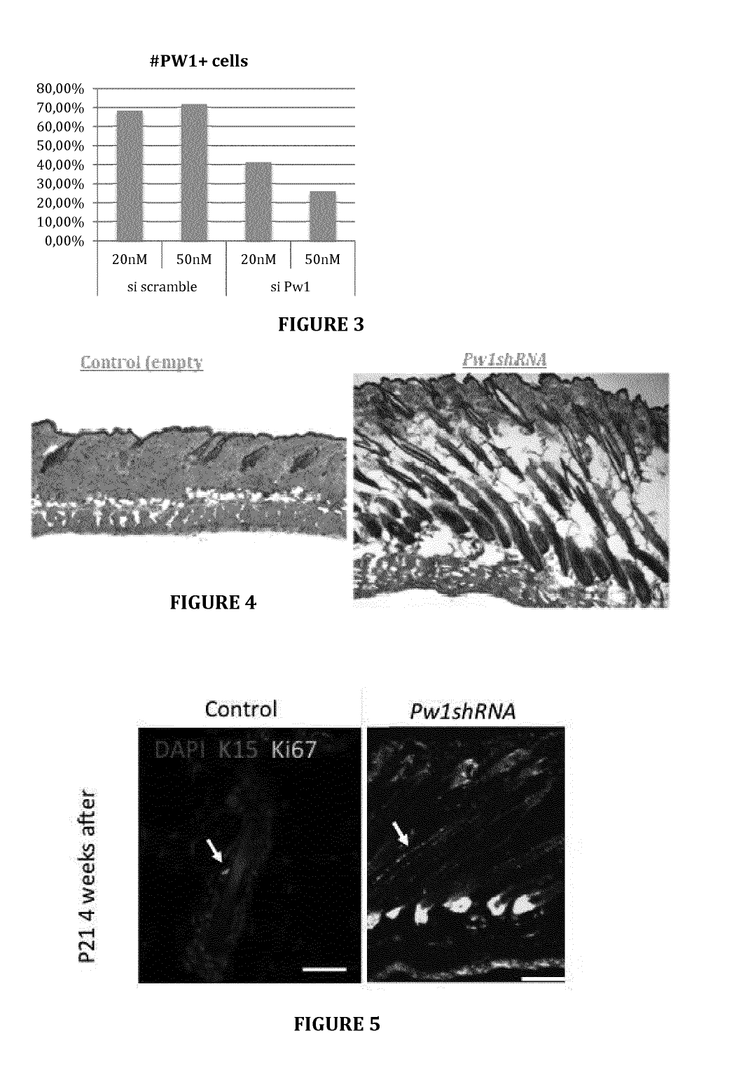

FIG. 3 shows a decrease of PW1 protein in neural stem cells transfected with siRNA s71469 (Ambion) that shows SEQ ID NO:3 as sense sequence.

Cells are plated at day 0 at a density of 20 000 cells/500 μl in OPTIMEM 1 medium provided by invitrogen without antibiotics. At day 1, cells are transfected using Forward transfection: Lipofectamine RNAiMAX invitrogen, following manufacturer instructions. 20 nM or 50 nM of siRNA were used. 50 nM allows 60% down regulation after 3 days of transfection.

### Example 2

### Activation of Hair Growth Cycle

Down-regulation of PW1 in the back of postnatal (P21) wild-type mice was performed by local injection between the dermis and the epidermis of the Pw1shRNA (100 μl of virus 400 MOI in PBS). 7 days after infection, 2 cm² around the injection point are collected and analyzed by histology. The same experiment was performed with the lentivirus control (scramble). Injection of PW1shRNA results in a clear activation of hair follicle stem cells (see FIG. 4). The effects are highly specific such that the non-stem cells that make up most of the tissue are not affected.

### Example 3

### Activation of Skin Stem Cells

The inventors have then tested the effects of the Pw1 shRNA lentivirus directly on mouse skin in vivo. Intradermal infection of the PW1shRNA (MOI 300 in PBS) has been performed in the back of wild-type mouse of 21 days of age. After 4 weeks, a clear activation of the follicles is observed that corresponds to a specific activation of the cell cycle as detected by ki67, which lights up dividing cells (FIG. 5). The effects are highly specific and only the stem cells (expressing PW1) and their immediate progeny are mobilized.

### Example 4

### Proliferation of Muscle Stem Cells

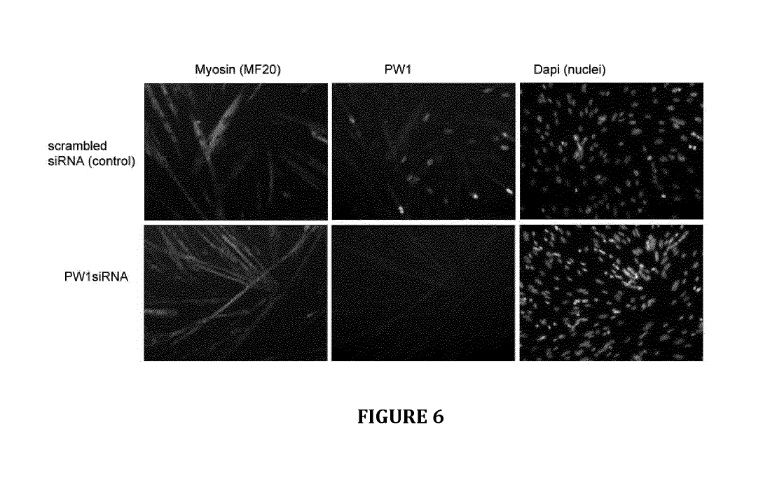

The inventors have further shown that siRNA directed to PW1 results in a clear down-regulation of PW1 in mouse muscle stem cells transfected with PW1 siRNA, as demonstrated on FIG. 6 by undetectable signal using immunofluorescence (PW1 antibody). Transfection with PW1 siRNA caused an increased cellular density resulting from increased levels of proliferation.

### REFERENCES

Allen, T. M. & Cullis, P. R. Drug delivery systems: entering the mainstream. Science 303, 1818-1822 (2004),
Dykxhoorn D. M., Novina C. D., Sharp P. A. (2003) Killing the messenger: short RNAs that silence gene expression. Nat Rev Mol Cell Biol. 4, 457-467.
Farokhzad, O. C. & Langer, R. Impact of nanotechnology on drug delivery. ACS Nano 3, 16-20 (2009)).
Green, H. Regeneration of the skin after grafting of epidermal cultures. Lab Invest 60, 583-584 (1989).
Higuchi Y, Kawakami S, Hashida M., BioDrugs. 2010 June; 24(3):195-205. Strategies for in vivo delivery of siRNAs: recent progress.
Kim, J., Ashworth, L., Branscomb, E., and Stubbs, L. (1997). The human homolog of a mouse-imprinted gene, Peg3, maps to a zinc finger gene-rich region of human chromosome 19q13.4. Genome Res 7, 532-540.
Kim, J., Bergmann, A., and Stubbs, L. (2000). Exon sharing of a novel human zinc-finger gene, ZIM2, and paternally expressed gene 3 (PEG3). Genomics 64, 114-118.
Oommen P. Varghese, Weilun Sun, Jons Hilborn and Dmitri A. Ossipov, In Situ Cross-Linkable High Molecular Weight Hyaluronan-Bisphosphonate Conjugate for Localized Delivery and Cell-Specific Targeting: A Hydrogel Linked Prodrug Approach, J Am Chem Soc. 2009 Jul. 1; 131(25):8781-3).
Soutschek J. et al. Nature. 2004 Nov. 11; 432(7014):173-8
Relaix F, Wei X J, Wu X, Sassoon D A. Nat Genet. 1998 March; 18(3):287-91.
Whitehead K A, Langer R, Anderson D G, Nat Rev Drug Discov. 2009 February; 8(2):129-38, Knocking down barriers: advances in siRNA delivery.
Yoo J W, Irvine D J, Discher D E, Mitragotri S Bio-inspired, bioengineered and biomimetic drug delivery carriers. Nat Rev Drug Discov. 2011 Jul. 1; 10(7):521-35).

1. A method of activating adult stem cells in a subject, the method comprising administering the subject with an interferent RNA that silences PW1/PEG3.

2. The method according to claim 1, wherein the interferent RNA is selected from the group consisting of a siRNA, a shRNA, an antisense RNA, and a miRNA.

3. The method according to claim 1, wherein the interferent RNA is a siRNA comprising a sense RNA strand and a complementary antisense RNA strand which together form an RNA duplex, wherein the sense RNA strand comprises or consists of sequence SEQ ID NO:3.

4. The method according to claim 1 wherein the interferent RNA is a shRNA.

5. The method according to claim 4-, wherein the interferent RNA is a shRNA that comprises or consists of sequence SEQ ID NO:14.

6. The method according to claim 1, wherein the subject is a patient afflicted with cardiac failure, bone marrow disease, skin disease, burns, degenerative disease such as diabetes, Alzheimer's disease, Parkinson's disease, or cancer.

7. A method for fighting against skin aging in a subject, the method comprising administering the subject with an interferent RNA that silences PW1/PEG3.

8. A method for preventing hair loss or promoting hair growth in a subject, the method comprising administering the subject with an interferent RNA that silences PW1/PEG3.

9. An in vitro method for activating adult stem cells, the method comprising contacting adult stem cells or a tissue comprising adult stem cells with an interferent RNA that silences PW1/PEG3.

10. The method according to claim 7, wherein the interferent RNA is selected from the group consisting of a siRNA, a shRNA, an antisense RNA, and a miRNA.

11. The method according to claim 7, wherein the interferent RNA is a siRNA comprising a sense RNA strand and a complementary antisense RNA strand which together form an RNA duplex, wherein the sense RNA strand comprises or consists of sequence SEQ ID NO:3.

12. The method according to claim 7, wherein the interferent RNA is a shRNA.

13. The method according to claim 12, wherein the interferent RNA is a shRNA that comprises or consists of sequence SEQ ID NO:14.

14. The method according to claim 8, wherein the interferent RNA is selected from the group consisting of a siRNA, a shRNA, an antisense RNA, and a miRNA.

15. The method according to claim 8, wherein the interferent RNAis a siRNA comprising a sense RNA strand and a complementary antisense RNA strand which together form an RNA duplex, wherein the sense RNA strand comprises or consists of sequence SEQ ID NO:3.

16. The method according to claim 8, wherein the interferent RNA is a shRNA.

17. The method according to claim 16, wherein the interferent RNA is a shRNA that comprises or consists of sequence SEQ ID NO:14.

18. The method according to claim 9, wherein the interferent RNA is selected from the group consisting of a siRNA, a shRNA, an antisense RNA, and a miRNA.

19. The method according to claim 9, wherein the interferent RNAis a siRNA comprising a sense RNA strand and a complementary antisense RNA strand which together form an RNA duplex, wherein the sense RNA strand comprises or consists of sequence SEQ ID NO:3.

20. The method according to claim 9, wherein the interferent RNA is a shRNA.

21. The method according to claim 20, wherein the interferent RNA is a shRNA comprises or consists of sequence SEQ ID NO:14.


SEQUENCE LISTING (14 sequences)

SEQ ID NO: 1 - protein, 1571 aa, Mus musculus
MYHHEDDTNSDMNSDDDMSRSGRETPPPRPSHAFGSERDLERRGRSRDVEPRDRWPYTRNPRSRLPQRDLSLPVMSRPHFGLDRDDDRRSMDYESRSQDAESYQNVVELKEDKKPQNPIQDNLENYRKLLSLGVQLAEDDRHSHMTQGHSSRSKRTAYPSTSRGLKPMPEAKKPSHRRGICEDESSHGVIMEKFIKDVARNPKSGRARELNERPPPRFPRPNDNWKDSSSSRRESVIQERGYEGSAFRGGFRFNADLASRSRALERKRRYHFDSDERGSGHEHKSCVRKKPFECGAEMRQAMSMGNLNSPSFSESQSIDFGANPYVCDECGRQFSVISEFVEHQIMHTRENLYEYGESFIHSVAVNEVQKGQGGGKRFECKECGETFSRSAALAEHRQIHAREYLAECRDQEDEETIMPSPTFSELQKMYGKDKFYECKVCKETFLHSSALIEHQKIHGRGNSDDRDNERERERDRLRARAREQRERERERERERELGEPFLTCPNFNEFRKMYRKDKIYECKVCGESFLHLSSLREHQKIHTRGNPFENKSRMCEETFVPSQSLRRRQKTYREKLFDFNNARDALMGNSDSSEHQKNRSRRNFFEGRGFEKPFVESQKSHTITRPPENKDDDKPFTISVNPNDKLKFPIMENGSQGKSYERSVIHSLGSAEAQKSHGGLGFSKPRPVAESSTQSSSSIYYPRAHSGGNTYEGKEYKDSIIHSLPAPRPLKRHRANDHIQCDEGGESSIYIPDIINKGRKIPAREDAYEGSSSSNYHTPNVSRAEPPSLSGESHDSKQDVTFSVPSSSVREHQKARAKKKYIEPRNNETSVIHSLPFGELLAGHRRAKFFECQECGEAFARRSELIEHQKIHDRERPSGSRHYERSVIRSLAPSDPQTSYAQERFIQEQVRKFRAFGQRSTTSNNLSVQKIYAQETFNAEEPHDKETHGQKIHDKEPYGKEPSGKEPHGDEPQDKEPLDQEMRSEEPHDDKPHGQEPHDDKPHGQEPHDDKPHGQEPHGDEPHGQEPHGDEPHDKEPIDQEMRSEEPHSEESHGDEPHGEESHGQEKVEDATIQASVSEEHQKDDAGDAIYECQDCGLGFTDLNDLTSHQDTHSRKALVDSREYAHSEVHAHSVSEFEKKCSGEKLYECPKCGESFIHSSLLFEHQRVHEQDQLYSVKACDDAFIALLPVRPRRNCTVERNPAVSGSAIRCRQCGQGFIHSSALNEHMRQHRDNEIMEQSELSDEIFIQGLALTEYQGSETEEKLFECTICGECFFTAKQLGDHHTKVHKDEPYEYGPSYTHASFLTEPLRKHIPLYECKDCGQSFLDDTVIAERMVFHPEREGGSEIVAATAQEVEANVLIPQEVLRIQGSNAEAAEPEVEAAEPEVEAAEPEVEAAEPNGEAEGPDGEAAEPDGEAEQPNGEAEQPNGDADEPDGAGIEDPEERADEPEEDVEEPEGDADEPDGADIEDPEEEGEDQEIEVEEPYYNCHECAETFASSSAFGEHLKSHASVIIFEPANAPGECSGYIERASTSAGGAEQADDKYFKCDVCGQLFNDRLSLARHQNSHTG

SEQ ID NO: 2 - protein, 1588 aa, Homo sapiens
MLPPKHLSATKPKKSWAPNLYELDSDLTKEPDVIIGEGPTDSEFFHQRFRNLIYVEFVGPRKTLIKLRNLCLDWLQPETRTKEEIIELLVLEQYLTIIPEKLKPWVRAKKPENCEKLVTLLENYKEMYQPEDDNNSDVTSDDDMTRNRRESSPPHSVHSFSDRDWDRRGRSRDMEPRDRWSHTRNPRSRMPPRDLSLPVVAKTSFEMDREDDRDSRAYESRSQDAESYQNVVDLAEDRKPHNTIQDNMENYRKLLSLVQLAEDDGHSHMTQGHSSRSKRSAYPSTSRGLKTMPEAKKSTHRRGICEDESSHGVIMEKFIKDVSRSSKSGRARESSDRSQRFPRMSDDNWKDISLNKRESVIQQRVYEGNAFRGGFRFNSTLVSRKRVLERKRRYHFDTDGKGSIHDQKGCPRKKPFECGSEMRKAMSVSSLSSLSSPSFTESQPIDFGAMPYVCDECGRSFSVISEFVEHQIMHTRENLYEYGESFIHSVAVSEVQKSQVGGKRFECKDCGETFNKSAALAEHRKIHARGYLVECKNQECEEAFMPSPTFSELQKIYGKDKFYECRVCKETFLHSSALIEHQKIHFGDDKDNEREHERERERERGETFRPSPALNEFQKMYGKEKMYECKVCGETFLHSSSLKEHQKIHTRGNPFENKGKVCEETFIPGQSLKRRQKTYNKEKLCDFTDGRDAFMQSSELSEHQKIHSRKNLFEGRGYEKSVIHSGPFTESQKSHTITRPLESDEDEKAFTISSNPYENQKIPTKENVYEAKSYERSVIHSLASVEAQKSHSVAGPSKPKVMAESTIQSFDAINHQRVRAGGNTSEGREYSRSVIHSLVASKPPRSHNGNELVESNEKGESSIYISDLNDKRQKIPARENPCEGGSKNRNYEDSVIQSVFRAKPQKSVPGEGSGEFKKDGEFSVPSSNVREYQKARAKKKYIEHRSNETSVIHSLPFGEQTFRPRGMLYECQECGECFAHSSDLTEHQKIHDREKPSGSRNYEWSVIRSLAPTDPQTSYAQEQYAKEQARNKCKDFRQFFATSEDLNTNQKIYDQEKSHGEESQGENTDGEETHSEETHGQETIEDPVIQGSDMEDPQKDDPDDKIYECEDCGLGFVDLTDLTDHQKVHSRKCLVDSREYTHSVIHTHSISEYQRDYTGEQLYECPKCGESFIHSSFLFEHQRIHEQDQLYSMKGCDDGFIALLPMKPRRNRAAERNPALAGSAIRCLLCGQGFIHSSALNEHMRLHREDDLLEQSQMAEEAIIPGLALTEFQRSQTEERLFECAVCGESFVNPAELADHVTVHKNEPYEYGSSYTHTSFLTEPLKGAIPFYECKDCGKSFIHSTVLTKHKELHLEEEEEDEAAAAAAAAAQEVEANVHVPQVVLRIQGLNVEAAEPEVEAAEPEVEAAEPEVEAAEPNGEAEGPDGEAAEPIGEAGQPNGEAEQPNGDADEPDGAGIEDPEERAEEPEGKAEEPEGDADEPDGVGIEDPEEGEDQEIQVEEPYYDCHECTETFTSSTAFSEHLKTHASMIIFEPANAFGECSGYIERASTSTGGANQADEKYFKCDVCGQLFNDRLSLARHQNTHTG

SEQ ID NO: 3 - DNA, 21 bp, Artificial Sequence
  siRNA
gagucgcagucaaucgauutt

SEQ ID NO: 4 - DNA, 21 bp, Artificial Sequence
  siRNA
aaucgauugacugcgacucag

SEQ ID NO: 5 - DNA, 21 bp, Artificial Sequence
  siRNA
ccaugguagaggcaacucatt

SEQ ID NO: 6 - DNA, 21 bp, Artificial Sequence
  siRNA
ugaguugccucuaccauggat

SEQ ID NO: 7 - DNA, 21 bp, Artificial Sequence
  siRNA
gaccagcuguauuccguaatt

SEQ ID NO: 8 - DNA, 21 bp, Artificial Sequence
  siRNA
uuacggaauacagcugguctt

SEQ ID NO: 9 - DNA, 58 bp, Artificial Sequence
  shRNA
ccggcctcttagatagtcctgtgaactcgagttcacaggactatctaagaggtttttg

SEQ ID NO: 10 - DNA, 58 bp, Artificial Sequence
  shRNA
ccggccctaatgacaagctgaaattctcgagaatttcagcttgtcattagggtttttg

SEQ ID NO: 11 - DNA, 58 bp, Artificial Sequence
  shRNA
ccgggccgagtcataccagaatgttctcgagaacattctggtatgactcggctttttg

SEQ ID NO: 12 - DNA, 58 bp, Artificial Sequence
  shRNA
ccggccactgtacgaatgcaaagatctcgagatctttgcattcgtacagtggtttttg

SEQ ID NO: 13 - DNA, 58 bp, Artificial Sequence
  shRNA
ccggcctccatttatatcccagatactcgagtatctgggatataaatggaggtttttg

SEQ ID NO: 14 - DNA, 19 bp, Artificial Sequence
  shRNA
actgtacgaatgcaaagat